(19) United States
(12) Patent Application Publication
(10) Pub. No.: US 2026/0191498 A1
(43) Pub. Date: Jul. 9, 2026

(54) ULTRASOUND MEASURING SYSTEMS AND METHODS WITH CROSS SECTIONAL COMPILATION

(71) Applicant: Provisio Medical, Inc., San Diego, CA (US)

(72) Inventors: Stephen Eric Ryan, San Diego, CA (US); Christopher Evan Ryan, San Diego, CA (US)

(21) Appl. No.: 19/129,588

(22) PCT Filed: Nov. 21, 2023
(86) PCT No.: PCT/US2023/080813

§ 371 Date: May 13, 2025

Related U.S. Application Data
(60) Provisional application No. 63/384,758, filed on Nov. 22, 2022.

(51) Int. Cl. A61B 8/08; A61B 8/00; A61B 8/12

(52) U.S. Cl. CPC A61B 8/0891; A61B 8/0858; A61B 8/12; A61B 8/4477

(57) ABSTRACT

An ultrasound system for measuring the dimensions of a structure, the system including a flexible body elongated along a longitudinal axis for insertion into the structure. At least one ultrasound transducer arranged on the flexible body and configured to transmit and receive ultrasound signals to and from the structure. The system is configured to calculate distances between each transducer and the structure based on the received ultrasound signals and to calculate multiple cross-sectional shapes of the structure based on the distances. The flexible body includes one or more electrical waveguides. The system causes electrical pulses to travel through the one or more waveguides and a conductive element through which the flexible body moves to make time domain reflectometry (TDR) distance measurements based on responsive impedance signals created by the conductive element. The system is configured so that the TDR distance measurements correspond to relative longitudinal positions of the flexible body.

### REFERENCE TO RELATED APPLICATIONS

This application claims the benefit of U.S. Provisional Application No. 63/384,758, filed Nov. 22, 2022, which is hereby incorporated by reference in its entirety.

### BACKGROUND

### Field of the Disclosure

The present disclosure relates generally to systems, methods, and devices that utilize ultrasound to gather dimensional and physiological information about structures such as fluid-filled body vessels.

### Description of Related Art

Obtaining and utilizing structural information about patients is a critical aspect of diagnosing and treating many medical conditions. For example, within the field of endovascular medicine, it is important to gain structural and physiological information about diseased blood vessels when selecting among interventional techniques such as angioplasty, stents, and/or surgery. Recent studies have illustrated that the predominate cause of endovascular treatment failure is inaccurate sizing of vessels or inadequate treatment to achieve the lumen dimensions desired over an entire stenotic lesion. An improperly selected, dimensioned, and/or positioned medical device (e.g., a stent) and/or treatment can lead to highly adverse outcomes including avoidable death. Typical techniques used for analyzing the structural features of blood vessels include angiography. However, angiography only provides limited and imprecise information about the size and morphology of blood vessels and often does not allow the physician to adequately assess the lesion prior to treatment. Recent studies have shown that outcomes are significantly improved through the use of more advanced, more accurate imaging techniques.

Some imaging catheters utilize ultrasound or optical technologies to provide a more accurate cross-sectional imaging that may then be interpreted by the physician to determine, among other characteristics, the dimensions of the lumen surrounding the catheter. For example, Intravascular Ultrasound (IVUS) and Optical Coherence Tomography (OCT) have been used in interventional diagnostic procedures to image blood vessels to locate and characterize atherosclerosis and other vessel diseases and defects.

IVUS and OCT images can be used to determine information about a vessel, including vessel dimensions, and is typically much more detailed than the information that is obtainable from traditional angiography images, which are generally limited to two-dimensional shadow images of the vessel lumen. The information gained from more accurate imaging techniques can be used to better assess physiological conditions, select particular procedures, and/or improve performance of the procedure.

While current IVUS and OCT systems provide additional and more detailed information compared to angiograms, these IVUS and OCT systems introduce significant additional time, cost and complexity into minimally-invasive procedures. The components of these systems (e.g., transducers, wires, imaging circuitry, fiber-optics, etc.) can occupy a large footprint within the blood vessel and must often be deployed independently and at separate times from interventional procedures (e.g., angioplasty). Further, the images produced by IVUS and OCT systems may not directly provide useful information about blood vessels and are typically subject to nonconforming interpretations of different physicians. Thus, there is a need for an improved and more efficient way to get needed information about a vessel or structure, particularly information about the diameter and multi-dimensional profile of a vessel or structure, while not sacrificing speed and footprint needed for timely, efficient, and effective treatment.

### SUMMARY

Embodiments of the present disclosure include a novel implementation of an ultrasound measurement probe to approximate the dimensions and/or shape(s) of fluid-filled structures. Some embodiments include an elongated flexible body such as a catheter with multiple ultrasound transducers arranged circumferentially about the catheter for generating and receiving ultrasound signals to and from surrounding structure. As the elongated flexible body is moved within or about a structure, the transducers collect data at different positions with respect to the structure. The individual measurements by themselves will reflect a limited resolution and accuracy of measured structures, their dimensions, and shape. In some embodiments, data from individual measurements is collectively analyzed and used to improve the resolution and accuracy of measurements, determinations of morphology and/or shape, and/or calculated dimensions of the structures. Features of individual measurements may be identified and correlated with each other across multiple measurements. These features may include structural and/or morphological features such as correlated shapes and dimensions (e.g., diameters) of a blood vessel.

In some embodiments, a correlated feature includes a radial or longitudinal segment of a blood vessel wall that follows a particular shape and/or particular dimensions. In some embodiments, curves are fitted to features of multiple measurements. For example, a radial slice of a blood vessel may be measured using a plurality of transducers arranged circumferentially around an imaging probe such as described in U.S. Pat. No. 10,231,701 filed Mar. 14, 2014 (the '701 Patent), the entire contents of which is herein incorporated by reference. Independent distance measurements between the transducers and structure may be pieced together to provide a cross-sectional representation of the blood vessel.

As the measurement probe moves with respect to the structure, either incidentally or by actuation, sets of distance measurements between the probe and structures are obtained and multiple cross-sections are correspondingly calculated. These cross sections are compared to identify corresponding features between each other. For example, it may be determined that the cross section obtained at one time is rotated or shifted by a certain amount compared to a cross section obtained at another time. After such a determination is made, imaging/calculations from the cross-sections may be combined to create high definition (HD) cross-sections. For example, multiple distance measurements from the different positions of the probe within the lumen may be fitted together to generate a more accurate fit. In some embodiments, multiple cross sections at different longitudinal positions along a structure are similarly combined to create a high definition three-dimensional fit to the structure.

For purposes of summarizing the disclosure and the advantages achieved over the prior art, certain objects and advantages of the disclosure are described herein. Not all such objects or advantages may be achieved in any particular embodiment. Thus, for example, those skilled in the art will recognize that the invention may be embodied or carried out in a manner that achieves or optimizes one advantage or group of advantages as taught herein without necessarily achieving other objects or advantages as may be taught or suggested herein.

In a first aspect, a method of ultrasound measuring is described herein. The method includes transmitting multiple sets of ultrasound signals from a plurality of ultrasound transducers of an ultrasound probe toward a structure, wherein each set is transmitted when the ultrasound probe is at a different position with respect to the structure, receiving responsive sets of ultrasound signals at the ultrasound transducers responsive to the respective sets of transmitted ultrasound signals, for each responsive ultrasound signal of the responsive sets, calculating a distance between the receiving ultrasound transducer and the structure that is substantially along a perpendicular between the ultrasound transducer and the structure, identifying a common feature across the sets of calculated distances, and determining a shape or size of the structure based on the common feature and the calculated distances.

In some embodiments, the different position is based on movement of the structure or a medium in which the ultrasound probe is positioned. In some embodiments, the different position is based on movement of blood in which the ultrasound probe is positioned. In some embodiments, the different position is based on movement of a blood vessel in which the ultrasound probe is positioned. In some embodiments, the different position is based on a mechanical actuation of the ultrasound transducers. In some embodiments, the mechanical actuation comprises a rotation of the ultrasound probe. In some embodiments, the mechanical actuation comprises a longitudinal movement of the ultrasound probe within the structure. In some embodiments, determining the shape or size comprises combining the sets of calculated distances and determining a cross-sectional shape based on interpolating across the combined sets of calculated distances. In some embodiments, identifying the common feature comprises determining multiple cross-sectional shapes, each shape based on a set of calculated distances, and identifying the common feature as a particular feature common across the multiple cross-sectional shapes. In some embodiments, the particular feature common across the multiple cross-sectional shapes is a centroid of each of the multiple cross-sectional shapes. In some embodiments, identifying the common feature comprises iterating through a plurality of positional offsets between the multiple cross-sectional shapes and identifying one or more offsets that minimize the differences between the multiple cross-sectional shapes. In some embodiments, identifying the common feature comprises using a correlation model that characterizes one or more common shapes across each of the multiple cross-sectional shapes. In some embodiments, the structure is a blood vessel into which the ultrasound probe is inserted, and wherein the determining the shape or size of the structure comprises determining a cross-sectional shape of the wall of the blood vessel and dimensions of the wall. In some embodiments, the structure is a blood vessel into which the ultrasound probe is inserted, and wherein the determining the shape or size of the structure comprises determining a three-dimensional shape of the blood vessel based on determining multiple cross-sectional shapes of the blood vessel at multiple longitudinal positions of the blood vessel.

In another aspect, an ultrasound system for measuring the dimensions of a structure is described herein. The system includes a flexible body elongated along a longitudinal axis and assembled for insertion into the structure, a plurality of ultrasound transducers arranged on the flexible body, and one or more processors. The one or more processors are programmed and configured to cause transmit multiple sets of ultrasound signals from the plurality of ultrasound transducers toward the structure, wherein each set is transmitted when the flexible body is at a different position with respect to the structure, receive responsive sets of ultrasound signals at the plurality of ultrasound transducers responsive to the respective sets of transmitted ultrasound signals, for each responsive ultrasound signal of the responsive sets, calculate a distance between the receiving ultrasound transducer and the structure that is substantially along a perpendicular between the transducer and the structure, identify a common feature across the sets of calculated distances, and determine a shape or size of the structure based on the common feature and the calculated distances.

In some embodiments, the different position is based on movement of the structure or a medium in which the flexible body is positioned. In some embodiments, the different position is based on movement of blood in which the flexible body is positioned. In some embodiments, the different position is based on movement of a blood vessel in which the flexible body is positioned. In some embodiments, the different position is based on a mechanical actuation of the plurality of ultrasound transducers. In some embodiments, the mechanical actuation comprises a rotation of the flexible body. In some embodiments, the mechanical actuation comprises a longitudinal movement of the flexible body within the structure. In some embodiments, determining the shape or size comprises combining the sets of calculated distances and determining a cross-sectional shape based on interpolating across the combined sets of calculated distances. In some embodiments, the identifying the common feature comprises determining multiple cross-sectional shapes, each shape based on a set of calculated distances, and identifying the common feature as a particular feature common across the multiple cross-sectional shapes. In some embodiments, the particular feature common across the multiple cross-sectional shapes is a centroid of each of the multiple cross-sectional shapes. In some embodiments, identifying the common feature comprises iterating through a plurality of positional offsets between the cross-sectional shapes and identifying one or more offsets that minimize the differences between the cross-sectional shapes. In some embodiments, the identifying of a common feature comprises using a correlation model that characterizes one or more common shapes across each of the multiple cross-sectional shapes. In some embodiments, the structure is a blood vessel, and wherein the determining the shape or size of the structure comprises determining a cross-sectional shape of the wall of the blood vessel and dimensions of the wall. In some embodiments, the structure is a blood vessel, and wherein the determining the shape or size of the structure comprises determining a three-dimensional shape of the blood vessel based on determining multiple cross-sectional shapes of the blood vessel at multiple longitudinal positions of the blood vessel.

In another aspect, an ultrasound system for measuring the dimensions of a structure is provided herein. The system includes a flexible body elongated along a longitudinal axis and assembled for insertion into the structure, a plurality of ultrasound transducers arranged on the flexible body and one or more processors. The one or more processors are programmed and configured to cause transmit multiple sets of ultrasound signals from the plurality of ultrasound transducers toward the structure, wherein each set is transmitted when the flexible body is at different rotational positions with respect to the structure, receive responsive sets of ultrasound signals at the plurality of ultrasound transducers responsive to the respective sets of transmitted ultrasound signals, for each responsive ultrasound signal of the responsive sets, calculate a distance between the receiving ultrasound transducer and the structure that is substantially along a perpendicular between the ultrasound transducer and the structure, identify the longest of each of the sets of calculated distances, and determine a shape or size of the structure based on the common feature and the calculated distances.

In some embodiments, the one or more processors are further programmed and configured to compare each of the longest of each of the sets of calculated distances among the different rotational positions identifying when the longest distance changes from a shorter distance to a longer distance and then back to a shorter distance, therefore identifying the longest axis among the sets of calculated distances among the rotational positions. In some embodiments, the different rotational positions are sequential.

In another aspect, an ultrasounds system for measuring the dimensions of a structure is provided herein. The system includes a flexible body elongated along a longitudinal axis and assembled for insertion into the structure, a plurality of ultrasound transducers arranged on the flexible body, and one or more processors. The one or more processors are programmed and configured to cause transmit multiple sets of ultrasound signals from the plurality of ultrasound transducers toward the structure, wherein each set is transmitted when the flexible body is at different rotational positions with respect to the structure, receive responsive sets of ultrasound signals at the plurality of ultrasound transducers responsive to the respective sets of transmitted ultrasound signals, for each responsive ultrasound signal of the responsive sets, calculate a distance between the receiving ultrasound transducer and the structure that is substantially along a perpendicular between the ultrasound transducer and the structure, identify the shortest of each of the sets of calculated distances, and determine a shape or size of the structure based on the common feature and the calculated distances.

In some embodiments, the one or more processors are further programmed and configured to compare each of the shortest of each of the sets of calculated distances among the different rotational positions identifying when the shortest distance changes from a longer distance to a shorter distance and then back to a longer distance, therefore identifying the shortest axis among the sets of calculated distances among the rotational positions. In some embodiments, the different rotational positions are sequential.

### BRIEF DESCRIPTION OF THE DRAWINGS

Embodiments of the disclosure will be described hereafter in detail with particular reference to the drawings. Throughout this description, like elements, in whatever embodiment described, refer to common elements wherever referred to and reference by the same reference number. The characteristics, attributes, functions, interrelations ascribed to a particular element in one location apply to that element when referred to by the same reference number in another location unless specifically stated otherwise. In addition, the exact dimensions and dimensional proportions to conform to specific force, weight, strength and similar requirements will be within the skill of the art after the following description has been read and understood.

All figures and drawn for ease of explanation of the basic teachings of the present disclosure only; the extensions of the figures with respect to number, position, relationship and dimensions of the parts to form examples of the various embodiments will be explained or will be within the skill of the art after the present disclosure has been read and understood.

FIG. 1 is an illustrative diagram of an ultrasound catheter probe system according to some embodiments.

FIG. 2A is an illustrative side perspective diagram of an ultrasound catheter probe placed within a lumen at different positions according to some embodiments.

FIG. 2B are cross-sectional perspective diagrams of the ultrasound catheter probe of FIG. 2A.

FIG. 3A is an illustrative diagram of an ultrasound catheter probe positioned within a lumen according to some embodiments.

FIG. 3B is an illustrative diagram of the ultrasound catheter probe of FIG. 3A repositioned within the lumen according to some embodiments.

FIGS. 4A, 4B, and 4C are illustrative diagrams of an ultrasound catheter probe positioned at multiple locations within a lumen according to some embodiments.

FIGS. 5A, 5B, and 5C are illustrative mappings of a lumen wall from ultrasound measurements obtained at the multiple locations shown in FIGS. 4A, 4B, and 4C, respectively.

FIG. 6 is an illustrative mapping of a lumen wall based on combining the mappings of FIGS. 5A, 5B, and 5C according to some embodiments.

FIG. 7A is an illustrative diagram of an ultrasound catheter probe repositioned at multiple longitudinal positions of a lumen and multiple positions within each cross section of the multiple longitudinal positions.

FIG. 7B is an illustrative diagram of a three-dimensional mapping of a lumen based on ultrasound measurements obtained at the multiple catheter probe positions of FIG. 7A.

FIG. 8A is an illustrative diagram of a curve-fitting applied to a set of calculated points of a lumen wall obtained from one position of an imaging probe according to some embodiments.

FIG. 8B is an illustrative diagram of a curve-fitting based on multiple sets of calculated points of a lumen wall obtained from multiple positions of an imaging probe according to some embodiments.

FIG. 9 is an illustrative flow chart of a process for determining the shape and size of a lumen based on multiple ultrasound measurements at multiple positions within the lumen according to some embodiments.

FIGS. 10A, 10B, and 10C are illustrative diagrams of an irregular lumen based on ultrasound measurements from multiple rotational orientations of an imaging probe according to some embodiments.

FIGS. 11A, 11B, and 11C are illustrative diagrams of an irregular lumen based on ultrasound measurements from multiple rotational orientations of an imaging probe according to some embodiments.

### DETAILED DESCRIPTION

In order that embodiments of the disclosure may be clearly understood and readily carried into effect, certain embodiments of the disclosure will now be described in further detail with reference to the accompanying drawings. The description of these embodiments is given by way of example only and not to limit the scope of the disclosure.

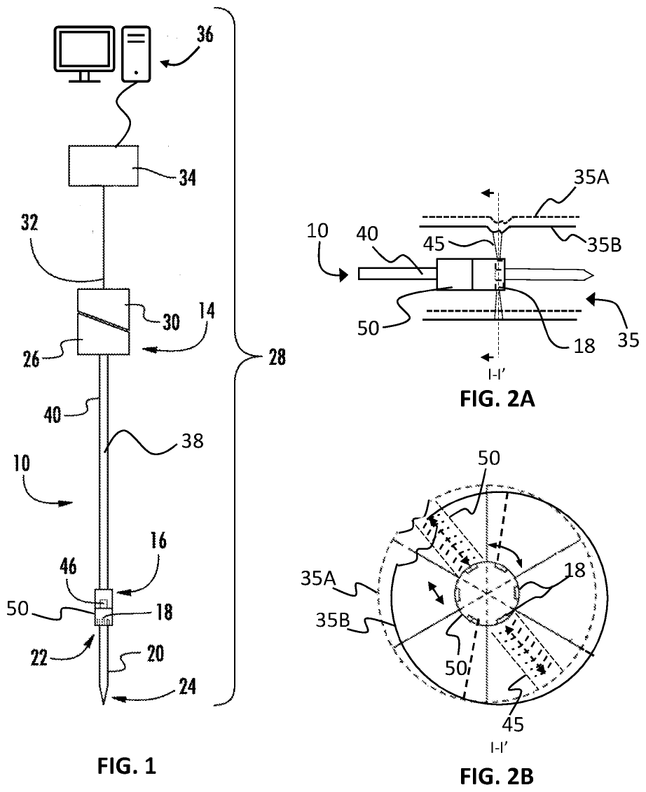

FIG. 1 is an illustrative diagram of an ultrasound catheter probe system 28 according to some embodiments. An ultrasound imaging probe 10 includes a body 40 having a proximal end 14 and a distal end 16. The probe 10 includes a plurality of transducers 18. Probe 10 also includes a body 40, an elongated tip 20 having a proximal end 22, and a distal end 24. Probe 10 includes a proximal connector 26 which connects probe 10 to other components of system 28, including a computer system 36. In an embodiment of the disclosure, the medical device 10 is part of a system 28 that includes a distal connector 30, electrical conductors 32, a data acquisition unit 34 and a computer system 36.

In some embodiments, body member 40 is tubular and has a central lumen 38. In some embodiments, body 40 has a diameter of about 650 μm or less. These dimensions are illustrative and not intended to be limiting. In some embodiments, the diameter of the probe 10 will depend on the type of device that probe 10 is integrated with and where the probe 10 will be used (e.g., in a blood vessel), which will become apparent to those of ordinary skill in the art in view of the present disclosure.

The proximal end 14 of the body 40 is attached to the proximal connector 26. In some embodiments, probe 10 and body 40 have an elongated tip 20 in which its proximal end 22 is attached to the distal end 16 of body 40. The elongated tip 20 may be constructed with an appropriate size, strength, and flexibility to be used for guiding probe 10 through a body lumen (e.g., a blood vessel). Elongated tip 20 and/or other components of probe 10 may include a radio-marker (e.g., visible to angiography) for precisely guiding the catheter through a lumen and positioning transducers 18 in the desired location. In some embodiments, probe 10 and distal end 16 are constructed and arranged for rapid exchange use. Body 40 and elongated tip 20 may be made of resilient flexible biocompatible material such as is common for IVUS and intravascular catheters known to those of ordinary skill in the art.

In some embodiments, probe 10 and body 40 may have multiple lumens for use with various features not shown (guidewires, fiberoptics, saline flush lumens, electrical connectors, etc.). In some embodiments, the outer diameter of the body 40 and elongated tip 20, if present, is substantially consistent along its length and does not exceed a predetermined amount.

In some embodiments, ultrasound transducers 18 are piezoelectric. The transducers may be built using piezoelectric ceramic or crystal material and layered by one or more matching layers that can be thin layers of epoxy composites or polymers. In some embodiments, the transducers are PMUTs (Piezoelectric Micromachined Ultrasonic Transducers), CMUTs (Capacitive Micromachined Ultrasonic Transducers), and/or photoacoustic transducers.

The operating frequency for the ultrasound transducers may be in the range of from about 8 to about 50 MHz or even up to about 60 MHz, depending on the dimensions and characteristics of the transducer and requirements of the particular application. Generally, higher frequency of operation provides better resolution and a smaller medical device 10. However, the tradeoff for this higher resolution and smaller catheter size may be a reduced depth of penetration into the tissue of interest and increased echoes from the blood itself (making the image more difficult to interpret). Lower frequency of operation is more suitable for imaging in larger vessels or within structures such as the chambers of the heart. Although specific frequency ranges have been given, this ranges given are illustrative and not limiting. The ultrasonic transducers 18 may produce and receive any frequency that leaves a transducer 18, impinges on some structure or material of interest and is reflected back to and picked up by a transducer 18.

The center resonant frequency and bandwidth of a transducer is generally related to the thickness of transducer materials generating or responding to ultrasound signals. For example, in some embodiments, a transducer includes a piezoelectric material such as quartz and/or lead-zirconate-titanate (PZT). A thicker layer will generally respond to a longer wavelength and lower frequency and vice versa. For example, a 50 micron thick layer of PZT will have a resonant frequency of about 40 MHz, a 65 micron thick layer will have a resonant frequency of about 30 MHz, and a 100 micron layer will have a resonant frequency of about 20 MHz. As further described herein, matching and backing layers may be included which affect the bandwidth and other characteristics of a transducer.

In some embodiments, probe 10 is connected with an actuating mechanism that may rotate and/or longitudinally move at least some portions of probe 10 and its transducers 18. A controlled longitudinal and/or radial movement permits the probe to obtain ultrasound readings from different perspectives within a surrounding structure, for example. Positioning the probe and its transducers in target locations may be augmented/guided by real-time imaging feedback provided by the transducers and system 28. Relative positions of the probe may be tracked and recorded during such processes (e.g., by using an encoder or other position sensing tool).

In some embodiments, system 28 is programmed to analyze and identify characteristics of the medium (e.g., blood) between probe 10 and structure in order to determine where the medium ends with respect to the structure (e.g., blood vessel wall). In some embodiments, multiple ultrasound measurements of the blood may be generated and the differences between the measurements are used to identify movement/change of the blood over time (e.g., as a result of a heart pumping). In some embodiments, doppler echo signals are used to determine these differences. Because the blood vessel wall does not have the same movement/change characteristics as the blood, the amount (or distance) between the probe 10 and blood vessel wall can be calculated. In some cases, reliance on the blood measurements without substantial reliance on measurements of the blood vessel wall may be used to determine the distance between probe 10 and blood vessel wall.

Computer system 36 is programmed to analyze and distinguish between the echoes associated with respective pulses. The computer system 36 is programmed to analyze the signals and calculate a radial distance measurement (e.g. D1, D2, . . . , D6) between each transducer 18 and lumen 35. This may be done, for example, by utilizing time-of-flight information of the echo signals and previously determined/differentiated signatures representative of a lumen wall (e.g., of lumen 35) and a particular medium (e.g., blood) between the transducer and lumen 35. Exemplary systems and methods for making such calculations are described, for example, in U.S. Pat. No. 10,231,701 filed Mar. 14, 2014 (the '701 Patent), the entire contents of which is herein incorporated by reference.

Based on distance calculations (D1, D2, . . . , D6), the shape and dimensions of lumen 35 may be estimated by further utilizing information including the dimensions of probe 10 and applying interpolation and/or other mathematical fitting techniques. For example, the relative positions of points (p1, . . . , p6) about lumen 35 may first be calculated and a curve fitting algorithm (e.g., spline interpolation) is applied to generate a two-dimensional slice representation of the lumen 35. As described in the '701 Patent, multiple slices can be calculated by taking sets ultrasound readings along the longitudinal extent of lumen 35 and combining them to generate a three-dimensional representation.

FIG. 2A is an illustrative side perspective diagram of an ultrasound catheter probe placed within a lumen at different positions according to some embodiments. FIG. 2B are cross-sectional perspective diagrams of the ultrasound catheter probe of FIG. 2A. Catheter probe 10 is shown inserted into a lumen 35. Lumen 35 is shown with lumen walls in a first position 35A and a second position 35B relative to probe 10. Shifting positions can result from movement of probe 10 (e.g., mechanical actuation) and/or movement of lumen walls between positions 35A and 35B (e.g., from heart pumping, blood flow).

Connected computer system 36 is programmed to cause transducers 18 to generate pulses 45 where each of the pulses is incident on different portions of lumen 35 substantially along a line perpendicular to the transducer. In response to echoes from lumen walls at positions 35A and 35B, transducers 18 generate electromagnetic signals respective to the pulses that reflect (i.e., echo) back from media and the lumen 35 adjacent and substantially perpendicular to probe 10. These electromagnetic signals are then processed by a signal processor and computer system 36. In some embodiments, an envelope signal associated with the activating pulse is detected and distinguished within the return signals to identify a transition between media and/or structural features. Based on the distinction, a distance measurement may be calculated between the transducer/probe and the transition location along a line substantially perpendicular to probe 10.

Other pulses may be similarly delivered/echoed using other transducers 18. In some embodiments, these pulses may be delivered simultaneously or at different times. Along with identifying and associating the signals with respective transducers, the computer system 36 is programmed to analyze the signals and calculate a radial distance measurement between each transducer 18 and lumen 35. This may be done, for example, by utilizing time-of-flight information of the echo signals and previously determined/differentiated signatures representative of a lumen wall (e.g., of a lumen wall represented at different times and positions 35A and 35B) and a particular medium (e.g., blood) between the transducer 18 and lumen walls at positions 35A and 35B. Exemplary systems and methods for making such calculations are described, for example, in U.S. Pat. No. 10,231,701 filed Mar. 14, 2014 (the '701 Patent), the entire contents of which is herein incorporated by reference.

Based on distance calculations, the shape and dimensions of the lumen may be estimated by further utilizing information including the dimensions of probe 10 and applying interpolation and/or other mathematical fitting techniques. For example, the relative positions of points about lumen walls at positions 35A and/or 35B may first be calculated and a curve fitting algorithm (e.g., spline interpolation) is applied to generate a two-dimensional slice representation of the lumen based on signals from the walls at positions 35A and 35B.

In some embodiments, each of the sets of points/cross-sections of a particular longitudinal lumen position over multiple time points/probe positions is analyzed to determine a common geometric feature among them. In some embodiments, a centroid for each set of points/cross section is determined.

In some embodiments, identifying the common feature includes iterating through a plurality of positional offsets between the cross-sectional shapes and identifying one or more positional offsets that minimize the differences between the cross-sectional shapes (e.g., using linear regression or other minimizing/matching technique known to those of skill in the art). In some embodiments, identifying of a common feature includes using another correlation model (e.g., based on a machine learning system such as a neural-network, K-nearest neighbor, Kernel estimation, Bayes classifier, Quadratic discriminant analysis, support vector machine, etc.) that characterizes one or more common shapes across each of the multiple cross-sectional shapes.

After determining a common centroid (or other correlation), the point sets/cross-sections are calibrated/offset to a common coordinate system with respect to their common feature/correlation. In some embodiments, a new curve (e.g., spline) is fitted based on all of the calibrated point sets/cross-sections to determine a refined shape of the cross-section of that particular longitudinal position of the lumen. Multiple cross-sections of the lumen at different longitudinal positions can be similarly combined and fitted to each other to determine a 3-dimensional profile/shape of the lumen.

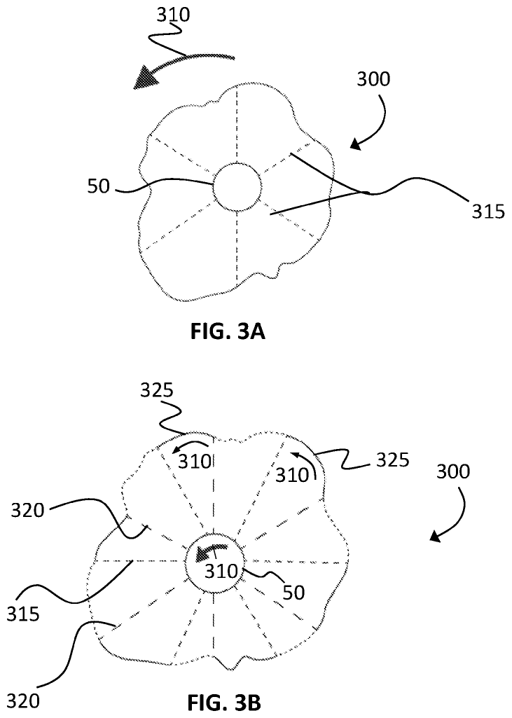

FIG. 3A is an illustrative diagram of an ultrasound catheter probe positioned within a lumen according to some embodiments. FIG. 3B is an illustrative diagram of the ultrasound catheter probe of FIG. 3A repositioned within the lumen according to some embodiments. In order to obtain multiple cross-sections from multiple positions, a catheter probe 50 is rotated by an amount 310 within a lumen 300. At a first position shown in FIG. 3A, a set of distance measurements along radial distance lines 315 are obtained between transducers of probe 50 and lumen 300 such as described herein. A first cross-sectional shape of lumen 300 may be calculated (e.g., using splines) based on the end-points of radial distance lines 315.

After repositioning/rotating probe 50 to a second position as shown in FIG. 3B, a second set of distance measurements are obtained along radial distance lines 320. A second cross-sectional shape of lumen 300 may be calculated based on the end-points of radial distance lines 320. A common feature (e.g., a centroid) between the cross-sectional shapes may be determined. As described herein, the point sets/cross-sections are calibrated/offset to a common coordinate system with respect to a common feature/correlation, from which a refined shape of the cross-section is determined. In some embodiments, multiple sets of radial distances are obtained at multiple rotational positions of probe 50 between the first and second positions over sections 325 as the probe is rotated.

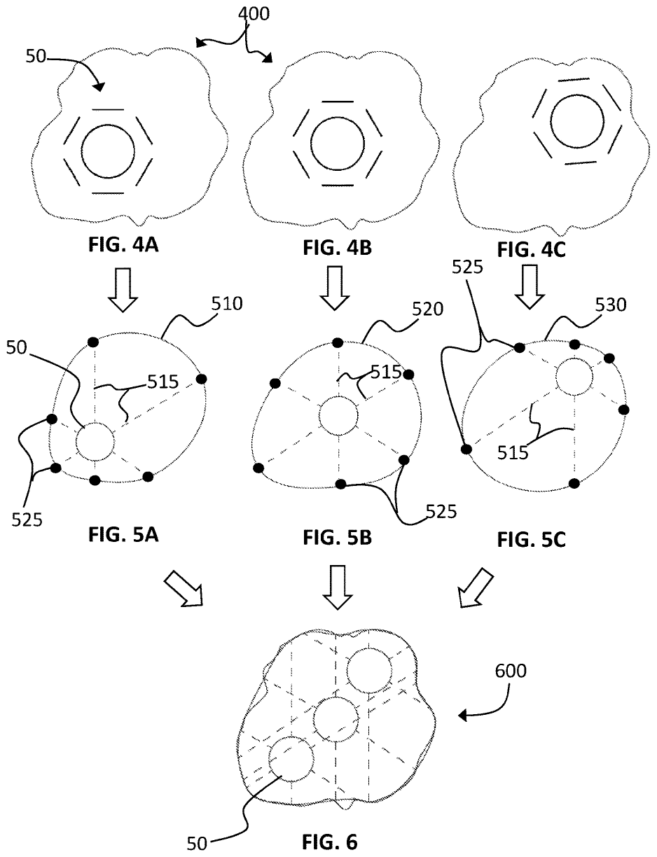

FIGS. 4A, 4B, and 4C are illustrative diagrams of an ultrasound catheter probe 50 positioned at multiple locations within a lumen 400 according to some embodiments. FIGS. 5A, 5B, and 5C are illustrative mappings of a lumen wall from ultrasound measurements obtained at the multiple locations shown in FIGS. 4A, 4B, and 4C, respectively. Catheter probe 50 is moved horizontally and/or vertically within a particular longitudinal cross-section of a lumen 400. At each of the respective positions of the probe 50, sets of distance measurements from transducers along perpendicular radial distance lines 515 to points 525 of lumen cross section 400 are obtained. Based on the respective sets of distance measurements and endpoints of calculated radial distance lines, shapes 510, 520, and 530 representing the lumen cross section are determined (e.g., using splines).

FIG. 6 is an illustrative mapping of a lumen wall based on combining the mappings of FIGS. 5A, 5B, and 5C according to some embodiments. After calculating shapes 510, 520, and 530, a common feature (e.g., a geometric feature such as a centroid) of the shapes is determined, and the shapes and endpoints are calibrated/offset according to a shared coordinate system based on the common feature. After calibration/offsetting, a refined shape 600 is calculated by combining information from the endpoints and/or shapes calculated for each set of measurements of the positions of probe 50. By using the combined information representing numerous data points of the lumen cross-section, the refined shape may more accurately represent the actual shape of the lumen cross section 400. Additional measurements taken at multiple positions between the positions shown in FIGS. 4A, 4B, and 4C and/or other positions (e.g., the rotational positions shown in FIGS. 3A and 3B) may be additionally combined to further calculate an enhanced/refined shape of the lumen cross-section.

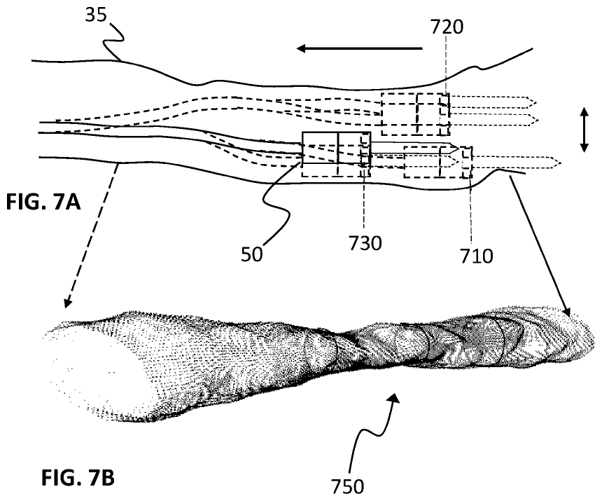

FIG. 7A is an illustrative diagram of an ultrasound catheter probe 50 repositioned at multiple longitudinal positions 710, 720, and 730 of a lumen and multiple positions within each cross section of the multiple longitudinal positions. In some embodiments, a probe is moved longitudinally within a lumen (e.g., by way of a “pullback” operation). At the different positions 710, 720, and 730, a set of distance measurements using the probe 50 is obtained (e.g., as described further herein). The different positions shown can additionally include different lateral positions within the same cross-section of a lumen (e.g., as shown in FIGS. 4A-4C) and/or rotational positions (e.g., as shown in FIGS. 3A and 3B).

FIG. 7B is an illustrative diagram of a three-dimensional mapping of a lumen based on ultrasound measurements obtained at the multiple catheter probe positions of FIG. 7A. Based on the multiple sets of measurements obtained, respective cross-sectional shapes of the lumen 35 may be calculated such as further described herein. Commonality of geometric features (e.g., centroids) may be determined with respect to the sets of measurements and/or cross-sectional shapes, based on which the sets are aligned with respect to each other. After aligning the sets of measurements with respect to each other, the sets and/or calculated cross-sectional shapes are combined to determine refined/enhanced cross-sectional shapes and/or a three-dimensional luminal shape 750 extending between the cross-sectional shapes.

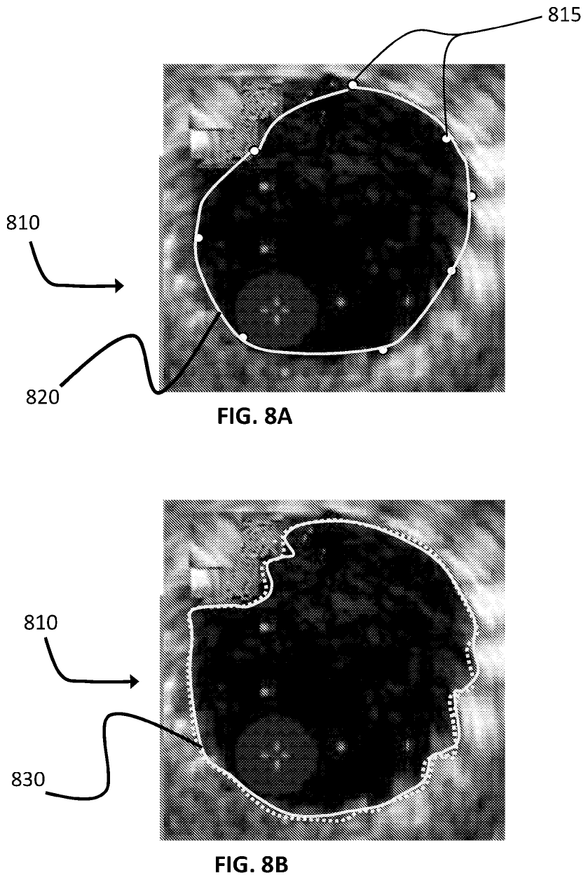

FIG. 8A is an illustrative diagram of a curve-fitting applied to a set of calculated points of a lumen wall 810 obtained from one position of an imaging probe according to some embodiments. FIG. 8A illustrates how an initial curve fit 820 to a single set of radial distance measurement points 815 may only provide a limited representation of a lumen wall that does not fully reflect abnormalities within the wall.

FIG. 8B is an illustrative diagram of a curve-fitting based on multiple sets of calculated points of a lumen wall 810 obtained from respective multiple positions of an imaging probe according to some embodiments. After calibrating/offsetting sets of points with respect to each other as further described herein, an enhanced curve-fitting (shown at 830) based on the combination of sets of points may be performed that further refines a correspondence between a curve fitting and the actual shape of the lumen.

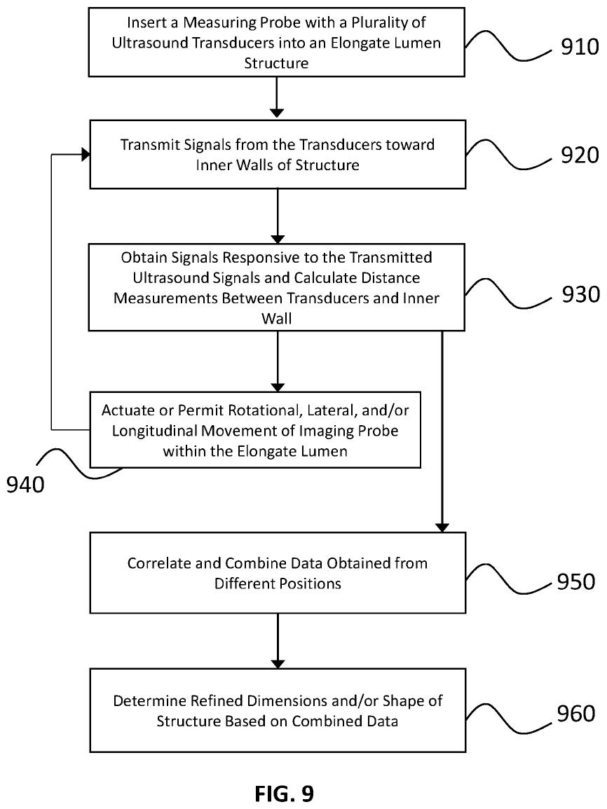

FIG. 9 is an illustrative flow chart of a process for determining the shape and size of a lumen based on multiple ultrasound measurements at multiple positions within the lumen according to some embodiments. At block 910, a measuring probe is inserted into a lumen such as in accordance with a percutaneous angioplasty procedure. The probe includes a plurality of ultrasound transducers (e.g., as shown in FIG. 1) arranged and configured to obtain signals representing the radial distance between each transducer and lumen wall within a cross-section of the lumen perpendicular to the probe (e.g., as shown in FIGS. 2A and 2B).

At block 920, at a particular longitudinal position within the lumen, the transducers are activated and transmit a series of radially directed signals toward the lumen wall within a cross-section of and perpendicular to the lumen. At block 930, signals are received back at each of the transducers representing distance measurements between each respective transducer and the lumen wall. Based on the received signals, distance measurements are calculated representing the distances between respective transducers and the lumen wall that are substantially within the cross-section and perpendicular to the lumen wall. Measurement data may be stored in computer memory such as for future analysis as described herein.

At block 940, the position of the probe may be actuated and/or permitted to change over an interval of time to a new position. The position of the probe may change rotationally, laterally, and/or longitudinally within a lumen (e.g., as shown in FIGS. 2A-2B, 3A-3B, 4A-4C, and 7A). Movement of the probe may occur through a motorized actuating mechanism (e.g., a rotary and/or linear actuator) connected with the probe, manual actuation such as by an operator pulling or twisting the proximate end of a catheter probe, and/or permitting incidental movement through blood flow, heart pumping, and/or other body movement such as over a predetermined interval of time. After movement to a new position within the lumen, the probe is again used to obtain another set of distance measurements at blocks 920 and 930. The probe may be further repositioned at block 940 and used to obtain additional sets of distance measurements.

At block 950, after multiple sets of distance measurements have been obtained at multiple positions within the lumen, the sets of distance measurements are collectively analyzed and used to determine a shape of the lumen at block 960. In some embodiments, a shape of a cross-section of the lumen is determined for each set of measurements (e.g., based on curve fitting the end-points of radial distance lines as further described herein). Among the sets of distance measurements, a correlated feature (e.g., a geometric feature such as a centroid) is determined and used to align the measurements (and calculated end-points of radial distance lines) with respect to each other according to a common coordinate system. In some embodiments, the shortest and/or longest of each set of calculated distances is identified. In some embodiments, one or more processors compare the longest of each of the sets of calculated distances among the multiple sequential rotational positions identifying when the longest distance changes from a shorter distance to a longer distance and then back to a shorter distance, therefore identifying the longest axis among the sets of calculated distances among rotational positions. In some embodiments, one or more processors compare each of the shortest of each of the sets of calculated distances among the multiple sequential rotational positions identifying when the shortest distance changes from a longer distance to a shorter distance and then back to a longer distance, therefore identifying the shortest axis among the sets of calculated distances among rotational positions.

In some embodiments, identifying the common feature includes iterating through a plurality of positional offsets between the cross-sectional shapes and identifying one or more positional offsets that minimize the differences between the cross-sectional shapes (e.g., using linear regression or other minimizing/matching technique known to those of skill in the art). In some embodiments, identifying of a common feature includes using another correlation model (e.g., based on a machine learning system such as a neural-network, K-nearest neighbor, Kernel estimation, Bayes classifier, Quadratic discriminant analysis, support vector machine, etc.) that characterizes one or more common shapes across each of the multiple cross-sectional shapes.

At block 960, after aligning the sets of measurements and/or respective shapes at block 950, one or more refined shapes are determined based on a combination of the sets of measurements/shapes (e.g., as shown in FIG. 6). For example, a new curve may be fit to multiple sets of co-aligned measurements of the same cross-section of a lumen to determine a refined curve/shape of the cross-section. In some embodiments, a curve is fit across multiple cross-sections (i.e., across a longitudinal segment) is performed to determine a three-dimensional shape of the lumen (e.g., as shown in FIG. 7B).

The processes described herein (e.g., the processes of FIG. 9) are not limited to use with the hardware shown and described herein. They may find applicability in any computing or processing environment and with any type of machine or set of machines that is capable of running a computer program. The processes described herein may be implemented in hardware, software, or a combination of the two. The processes described herein may be implemented in computer programs executed on programmable computers/machines that each includes a processor, a non-transitory machine-readable medium or other article of manufacture that is readable by the processor (including volatile and non-volatile memory and/or storage elements), at least one input device, and one or more output devices. Program code may be applied to data entered using an input device to perform any of the processes described herein and to generate output information.

The processing blocks (for example, in the processes of FIG. 9) associated with implementing the system may be performed by one or more programmable processors executing one or more computer programs to perform the functions of the system. All or part of the system may be implemented as, special purpose logic circuitry (e.g., an FPGA (field-programmable gate array) and/or an ASIC (application-specific integrated circuit)). All or part of the system may be implemented using electronic hardware circuitry that include electronic devices such as, for example, at least one of a processor, a memory, a programmable logic device, and/or a logic gate.

The processes described herein are not limited to the specific examples described. For example, the process of FIG. 9 are not limited to the specific processing orders illustrated. Rather, any of the processing blocks of FIG. 9 may be re-ordered, combined or removed, performed in parallel or in serial, as necessary, to achieve the results set forth above.

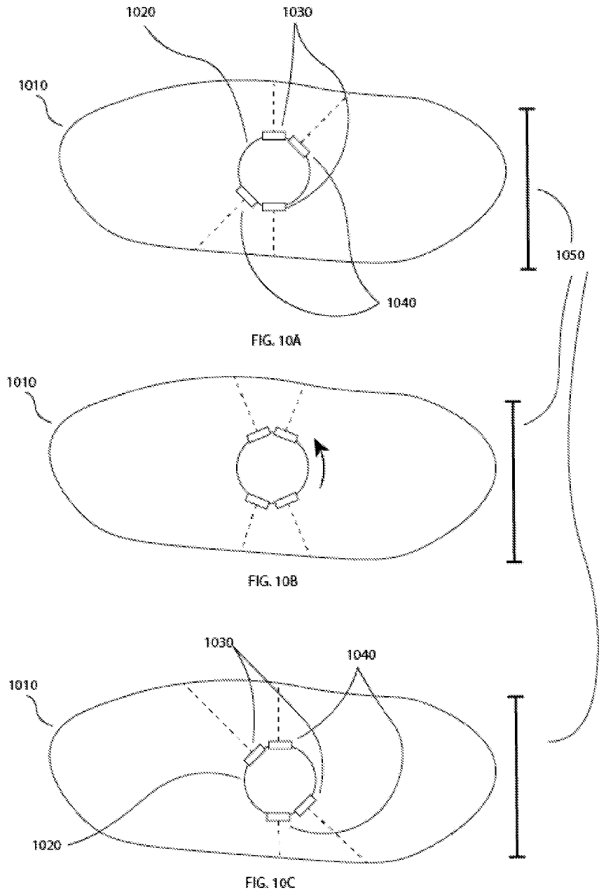

FIG. 10A is an illustrative diagram of an irregular lumen based on ultrasound measurements from multiple rotational orientations of an imaging probe according to some embodiments. When a probe 1020 is placed in a venous lumen 1010 it obtains ultrasound measurements. FIG. 10B is an illustrative diagram of probe 1020 being rotated with an irregular venous lumen 1010. The probe 1020 can then be rotated by a physician to obtain additional measurements of the vessel lumen. As the probe 1020 is rotated transducer set 1030 continues to take measurements of the vessel lumen starting from measuring the minimum diameter 1050. FIG. 10C is an illustrative diagram of a probe 1020 which has been rotated within an irregular vessel lumen 1010. So long as the longitudinal position of the probe 1020 does not substantially change during rotation, transducer set 1040 will measure the minimum vessel lumen diameter after sufficient rotation as shown in FIG. 10C. Given that transducers are placed circumferentially about the probe 1020 at regular intervals a minimum diameter measurement 1050 by transducer set 1040 indicates the probe 1020 has been rotated sufficiently to measure each point of the vessel lumen. These individual measurements taken during the rotation of the probe 1020 by each set of transducers can then be compiled by a computer program 36 to create a measurement compilation of a single longitudinal splice of the vessel lumen 1010.

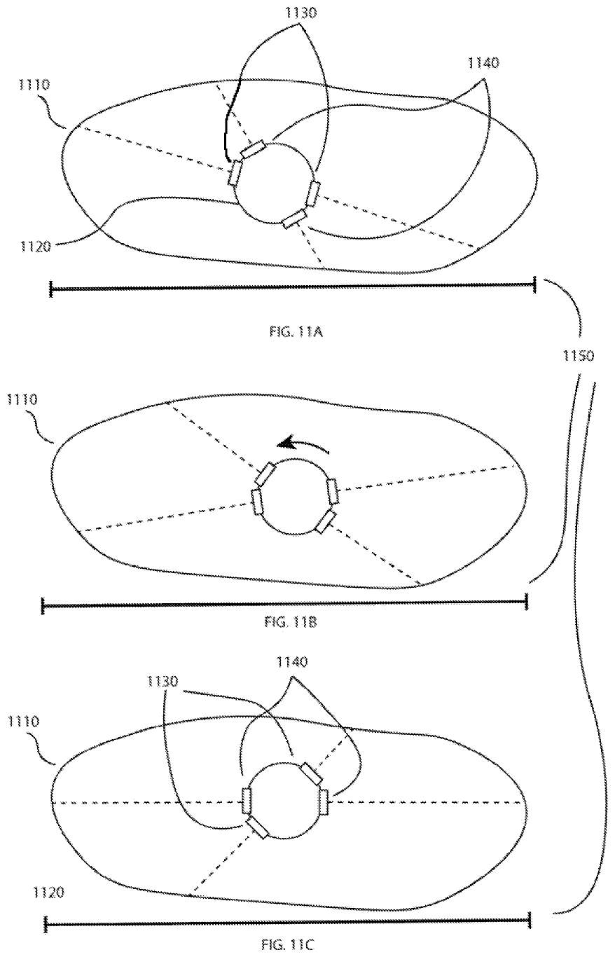

FIG. 11A is an illustrative diagram of an irregular lumen based on ultrasound measurements from multiple rotational orientations of an imaging probe according to some embodiments. When a probe 1120 is placed in a venous lumen 1110 it obtains ultrasound measurements. Transducer set 1130 takes ultrasound readings of the venous lumen 1110 relatively near the maximum diameter of the vessel lumen. FIG. 11B is an illustrative diagram of probe 1020 being rotated with an irregular venous lumen 1110. The probe 1120 can then be rotated by a physician to obtain additional measurements of the vessel lumen. As the probe 1120 is rotated transducer set 1130 continues to take measurements of the vessel lumen starting just before from measuring the maximum diameter 1150. After transducer set 1130 is rotated past the maximum diameter the diameter measured begins to decrease. This maximum diameter threshold is the inflection point between increasing diameter ultrasound measurements and decreasing diameter ultrasound measurements. FIG. 11C is an illustrative diagram of a probe 1120 which has been rotated within an irregular vessel lumen 1110. So long as the longitudinal position of the probe 1120 does not substantially change during rotation, transducer set 1140 will measure the maximum vessel lumen diameter after sufficient rotation as shown in FIG. 11C. Sufficient rotation can be determined after transducer set 1140 has also past the inflection point from between increasing diameter ultrasound measurements and decreasing diameter ultrasound measurements. Given that transducers are placed circumferentially about the probe 1120 at regular intervals a maximum diameter measurement 1150 by transducer set 1140 indicates the probe 1120 has been rotated sufficiently to measure each point of the vessel lumen. These individual measurements taken during the rotation of the probe 1120 by each set of transducers can then be compiled by a computer program 36 to create a measurement compilation of a single longitudinal splice of the vessel lumen 1110.

Elements of different embodiments described herein may be combined to form other embodiments not specifically set forth above. Various elements, which are described in the context of a single embodiment, may also be provided separately or in any suitable subcombination. Other embodiments not specifically described herein are also within the scope of the following claims.

1-14. (canceled)

15. An ultrasound system for measuring the dimensions of a structure, the system comprising:
a flexible body elongated along a longitudinal axis and assembled for insertion into the structure;
a plurality of ultrasound transducers arranged on the flexible body; and
one or more processors programmed and configured to cause:
transmit multiple sets of ultrasound signals from the plurality of ultrasound transducers toward the structure, wherein each set is transmitted when the flexible body is at a different position with respect to the structure;
receive responsive sets of ultrasound signals at the plurality of ultrasound transducers responsive to the respective sets of transmitted ultrasound signals;
for each responsive ultrasound signal of the responsive sets, calculate a distance between the receiving ultrasound transducer and the structure that is substantially along a perpendicular between the transducer and the structure;
identify a common feature across the sets of calculated distances; and
determine a shape or size of the structure based on the common feature and the calculated distances.

16. The system of claim 15, wherein the different position is based on movement of the structure or a medium in which the flexible body is positioned.

17. The system of claim 15, wherein the different position is based on movement of blood in which the flexible body is positioned.

18. The system of claim 15, wherein the different position is based on movement of a blood vessel in which the flexible body is positioned.

19. The system of claim 15, wherein the different position is based on a mechanical actuation of the plurality of ultrasound transducers.

20. The system of claim 19, wherein the mechanical actuation comprises a rotation of the flexible body.

21. The system of claim 19, wherein the mechanical actuation comprises a longitudinal movement of the flexible body within the structure.

22. The system of claim 15, wherein determining the shape or size comprises combining the sets of calculated distances and determining a cross-sectional shape based on interpolating across the combined sets of calculated distances.

23. The system of claim 15, wherein the identifying the common feature comprises determining multiple cross-sectional shapes, each shape based on a set of calculated distances, and identifying the common feature as a particular feature common across the multiple cross-sectional shapes.

24. The system of claim 23, wherein the particular feature common across the multiple cross-sectional shapes is a centroid of each of the multiple cross-sectional shapes.

25. The system of claim 23, wherein identifying the common feature comprises iterating through a plurality of positional offsets between the cross-sectional shapes and identifying one or more offsets that minimize the differences between the cross-sectional shapes.

26. The system of claim 23, wherein the identifying of a common feature comprises using a correlation model that characterizes one or more common shapes across each of the multiple cross-sectional shapes.

27. The system of claim 15, wherein the structure is a blood vessel, and wherein the determining the shape or size of the structure comprises determining a cross-sectional shape of the wall of the blood vessel and dimensions of the wall.

28. The system of claim 15, wherein the structure is a blood vessel, and wherein the determining the shape or size of the structure comprises determining a three-dimensional shape of the blood vessel based on determining multiple cross-sectional shapes of the blood vessel at multiple longitudinal positions of the blood vessel.

29. An ultrasound system for measuring the dimensions of a structure, the system comprising:
a flexible body elongated along a longitudinal axis and assembled for insertion into the structure;
a plurality of ultrasound transducers arranged on the flexible body; and
one or more processors programmed and configured to cause:
transmit multiple sets of ultrasound signals from the plurality of ultrasound transducers toward the structure, wherein each set is transmitted when the flexible body is at different rotational positions with respect to the structure;
receive responsive sets of ultrasound signals at the plurality of ultrasound transducers responsive to the respective sets of transmitted ultrasound signals;
for each responsive ultrasound signal of the responsive sets, calculate a distance between the receiving ultrasound transducer and the structure that is substantially along a perpendicular between the ultrasound transducer and the structure;
identify the longest of each of the sets of calculated distances; and
determine a shape or size of the structure based on the common feature and the calculated distances.

30. The system of claim 29, wherein the one or more processors are further programmed and configured to compare each of the longest of each of the sets of calculated distances among the different rotational positions identifying when the longest distance changes from a shorter distance to a longer distance and then back to a shorter distance, therefore identifying the longest axis among the sets of calculated distances among the rotational positions.

31. The system of claim 30, wherein the different rotational positions are sequential.

32. An ultrasound system for measuring the dimensions of a structure, the system comprising:
a flexible body elongated along a longitudinal axis and assembled for insertion into the structure;
a plurality of ultrasound transducers arranged on the flexible body; and
one or more processors programmed and configured to cause:
transmit multiple sets of ultrasound signals from the plurality of ultrasound transducers toward the structure, wherein each set is transmitted when the flexible body is at different rotational positions with respect to the structure;
receive responsive sets of ultrasound signals at the plurality of ultrasound transducers responsive to the respective sets of transmitted ultrasound signals;
for each responsive ultrasound signal of the responsive sets, calculate a distance between the receiving ultrasound transducer and the structure that is substantially along a perpendicular between the ultrasound transducer and the structure;
identify the shortest of each of the sets of calculated distances; and
determine a shape or size of the structure based on the common feature and the calculated distances.

33. The system of claim 32, wherein the one or more processors are further programmed and configured to compare each of the shortest of each of the sets of calculated distances among the different rotational positions identifying when the shortest distance changes from a longer distance to a shorter distance and then back to a longer distance, therefore identifying the shortest axis among the sets of calculated distances among the rotational positions.

34. The system of claim 33, wherein the different rotational positions are sequential.
